(19) United States
(12) Patent Application Publication
(10) Pub. No.: US 2016/0278872 A1
(43) Pub. Date: Sep. 29, 2016

(54) End Effector For A Surgical Instrument And Surgical Instrument Comprising An End Effector

(71) Applicant: GOMTEC GMBH, Seefeld (DE)

(72) Inventors: Bernd Gombert, Worthsee (DE); Patrick Rothfuss, Hallbergmoos (DE); Leopold Bock-Krausen, Munchen (DE)

(21) Appl. No.: 15/021,715

(22) PCT Filed: Sep. 10, 2014
(86) PCT No.: PCT/EP2014/069282

§ 371 Date: Mar. 14, 2016

(30) Foreign Application Priority Data
Sep. 17, 2013 (DE) 10 2013 110 216.6

(51) Int. Cl. A61B 34/00; A61B 34/30; A61B 17/29

(52) U.S. Cl. CPC A61B 34/72; A61B 17/29; A61B 34/30; A61B 2017/2944; A61B 2017/00075

(57) ABSTRACT

The invention relates to an end effector (7) for a surgical instrument (1), said end effector (7) comprises a drive unit (6) comprising an electric motor (12) which rotationally drives a shaft (14, 18). According to the invention, the end effector (7) also comprises a rotation-translation-transmission (29) which is connected to the shaft (14, 18) and which translates a rotating movement of the shaft (14, 18) into a translatory movement, and at least one working element (8) which is coupled to the rotation-translation-transmission (29) and is only driven thereby in a translatory manner.

The invention relates to an end effector for a surgical instrument as well as a surgical instrument having an end effector.

Surgical interventions on human bodies are today carried out to an increasing extent with minimally invasive methods with the support of surgery robots. Depending on the type of intervention, the surgery robots can be equipped with various surgical instruments, such as, for example, endoscopes, cutting, gripping or sewing instruments. During an operation, the instruments are introduced into the body of the patient by means of one or more robots via a sheath. During the operation, the surgical instrument is then controlled by a surgeon via an input device of the robot system, such as, for example, via a joystick or by means of gesture control.

Today, a wide range of instruments are used for surgical use, such as, for example, endoscopes, laparoscopic instruments, cutting, gripping, holding, connecting or sewing instruments as well as other surgical tools. The actual end effector, such as, for example, a scalpel, scissors, a needle, a scraper, a file, a gripper, etc., is located at the distal end of the surgical instruments or tools. The surgical instruments known from prior art are typically actuated by means of a cable drive.

FIG. 1 shows the distal end of a surgical instrument 1 known from U.S. Pat. No. 6,312,435 which is designed for robot-supported minimally invasive surgery. The instrument 1 comprises a shaft 3 which extends in a longitudinal direction L and on the distal end of which the actual end effector 5, in the present case so-called Potts Scissors, is fastened pivotably. The scissors comprise two scissor blades which can be moved back and forth around the axis A1. The entire end effector 5 can additionally be pivoted around a pivot axis A2 running transversely to the axis A1. The surgical instrument 1 can furthermore be rotated around its longitudinal axis L. In this embodiment, the individual joints are each moved with the aid of a cable drive (not shown). The construction and drive mechanism of such a surgical instrument are, however, relatively elaborate and complicated.

It is therefore the object of the present invention to create a surgical instrument which is constructed clearly more simply than known surgical instruments with a cable drive. Additionally, an object of the present invention is to create an end effector having an integrated drive unit.

This object is solved according to the invention by the features specified in the independent claims. Further embodiments of the invention arise from the sub-claims.

According to the invention, an end effector for a surgical instrument is proposed which comprises a drive unit having an electric motor which drives a shaft in a rotational manner. The drive unit furthermore comprises a rotation-translation transmission which translates a rotational movement of the shaft into a translational movement and acts on a working element, such as, for example, a gripper, such that it is driven by the transmission in a translational manner, preferably in a purely translational manner. Such an end effector is therefore constructed to be substantially simpler than an end effector having a cable drive. According to the invention, the end effector furthermore comprises a repelling component which drives the working element in a closing direction.

The translational movement of the driven working element preferably runs transversely to a rotational axis, around which the shaft of the drive unit rotates.

A working element can, for example, be the jaws of a gripper, a scalpel, a scissor blade, a needle, a clamp or any other element of a known medical tool. Differently shaped working elements can also be present in an end effector; for example, the end effector can be formed as so-called anvil scissors, in which one working element is formed as a cutting element and the other as an anvil which complements the cutting element.

Furthermore, sensors can be attached/integrated to and/or into the working elements. Sensors which typically detect pressure, force, torque, temperature, acceleration/speed, distance. Imaging sensors are, however, also conceivable, as they are already available today in small versions in so-called image processors. These then not only serve for informative support of the surgeon, but also serve the surgical instrument and/or robot systems for the open-loop-control or closed-loop-control thereof. In particular, the joining of several sensors (keyword sensor fusion) also allows the operator or the surgical robot system to make decisions which support him in his work and/or also serve above all, however, for critical assessment/analysis of the functional safety. The task of such sensors would, however, also be to detect errors in the system or operating errors, electrical, software and/or mechanical failures and/or foreign influences such as collisions and/or to evaluate them according to a safety plan, to weight them and to initiate corresponding actions.

A further field of application of these sensors would also be to enable a tracking and recognition system for the gripper and/or the instrument. In particular, the positional and temporal sensory detection in Cartesian dimensions enables not only the detection of collisions/prevention of collisions, but additionally computer-supported and/or model-based collision consideration. The possibility to calculate this proactively therefore also enables a very early warning and therefore, as a consequence, a collision prevention strategy. This would mean that the surgical operator not only receives an assistance function “to hand”, but also the possibility of an automatic non-stop device. Therefore, in the preliminary stage, collisions can already be prevented which would no longer have been able to be prevented otherwise by the operator. Assistance, as well as automatic emergency and aid functions, can therefore be affixed/implemented not only locally, so in the end effector itself, but also in the instrument and/or in the robot system.

The end effector according to the invention preferably comprises means for releasable fastening of the end effector to the shaft of a surgical instrument. The end effector can therefore be exchanged or maintained simply. To fasten the end effector to a surgical instrument, for example, a screw or plug connection can be provided which can have, if necessary, catching means. The end effector can, however, also be mounted to be fixed to the surgical instrument.

According to a preferred embodiment of the invention, the rotation-translation transmission comprises a rotatable element having a planar curve which engages with at least one working element, which is driven in a translational manner, and guides this. The rotating element is preferably provided on an end of the shaft driven by an electric motor and can, for example, be designed in a disc shape.

The curve provided on the rotating element can, for example, be formed as a spiral-shaped thread path or as a spiral-shaped groove.

The curve preferably spans a flat surface, the surface normal of which is directed in the direction of the rotational axis of the shaft driven by an electric motor.

According to a specific embodiment of the invention, the rotation-transformation transmission can also comprise several curves which each engage with at least one working element and drive this differently. Therefore, for example, several drive elements can be driven with different translation. Alternatively or additionally, several working elements could also be driven at staggered times, so independently of one another.

One embodiment of the end effector according to the invention comprises at least one first working element which engages with a first curve of the rotation-translation transmission, and at least one second working element which engages with a second curve. The at least one first working element can therefore be driven using a first movement profile, and the at least one second working element using a, if necessary, different second movement profile. Such an embodiment of an end effector can, for example, comprise two first working elements which are guided by a first curve, and a second working element which is guided by a second curve.

According to a specific embodiment of the invention, the end effector comprises two working elements which are arranged opposite each other with respect to the rotational axis of the shaft and can be moved towards each other or away from each other by a rotational movement of the shaft. Both working elements thereby preferably engage with the same curve.

The rotating element of the rotation-translation transmission is preferably designed as a separate component which can be brought into engagement with the shaft of the drive unit. A sprocket can be provided on the end of the shaft which can engage with a corresponding recess on the rotating element.

Preferably, the rotating element of the rotation-translation transmission is pre-tensioned such that the working elements, for example in the event of failure of the drive unit, can close automatically. For example, a repelling component, for example a (spiral) spring, which is supported on the housing inner wall of the end effector, can act on the rotating element. The spring is tensioned by a rotational movement of the rotatable element in the opening direction of the end effector and therefore counteracts the drive unit. The spring can also be integrated into the end effector by pre-tensioning such that, even in the closed state of the end effector, a force of the spring is exerted in the closing direction. By relaxing the spring, the spring drives the rotating element in the closing direction of the end effector. The spring force is advantageously selected in such a way that frictional losses as well as opposing moments of the motor can be overcome.

Preferably, the actual tool of the end effector is designed as a separate component which can be fastened releasably to the drive unit of the end effector. In this case, the tool of the end effector preferably comprises a fastening device, such as, for example, a screw, plug or catch connection. The drive unit and the end effector could, however, also be formed in one piece.

According to a preferred embodiment, the whole end effector together with its drive unit is able to be mounted on the shaft of a surgical instrument. For this purpose, a suitable connection, for example a screw, plug or catch connection, or any other known quick connection mechanism, such as, for example, a bayonet catch, can in turn be provided.

The end effector according to the invention can also comprise a second drive unit with which the end effector can be rotated around the rotational axis of the shaft. The operating possibilities of the surgical instrument can thereby be further improved.

A particularly simple embodiment of an end effector results if the first drive unit to actuate the tool or working element of the end effector is constructed identically to the second drive unit to rotate the end effector.

The drive units according to the invention preferably each comprise an electric motor. The drive unit can furthermore comprise a transmission with which the rotational movement of the shaft driven by the electric motor is transferred to a second shaft.

The invention also relates to a surgical instrument for use on a surgery robot for minimally invasive surgery. The surgical instrument has a shaft which extends in the longitudinal direction of the surgical instrument, wherein an end effector is provided at the distal end of the shaft, as was described above.

The surgical instrument according to the invention can additionally have a manipulator to position the end effector, said manipulator having several rotatable elements. The manipulator can, for example, comprise at least one first rotatable element which is arranged to be rotatable around a first rotational axis, as well as at least one second rotatable element which is arranged to be rotatable around a second rotational axis. Additionally, the manipulator comprises a first drive unit to drive the first rotatable element and a second drive unit to drive the second rotatable element. The drive units are integrated into the manipulator. Furthermore, the first and second rotational axes are arranged at an angle to each other. Due to the angled arrangement of the rotational axes, it is possible to drive the rotatable elements directly, each with the aid of an electric motor, without having to divert the rotational movement of the electric motor via a cable mechanism to the pivot axes.

In an advantageous embodiment of the invention, the drive units of the manipulator and the drive unit of the end effector are constructed identically.

### SHORT DESCRIPTION OF THE DRAWINGS

The invention is explained in more detail below by way of example by means of the enclosed drawings. Here are shown:

FIG. 1 a surgical instrument known from prior art;

FIG. 2a a schematic side view of the tool of an end effector having two working elements;

FIG. 2b a schematic view of the end effector of FIG. 2a which has a rotatable element of a rotation-translation transmission having a single drive curve;

FIG. 3a a schematic side view of an end effector having three working elements;

FIG. 3b a schematic view of the end effector of FIG. 3a which has two drive curves of a rotation-translation transmission having two rotatable elements;

FIG. 4 a view of the tool of an end effector having mechanical as well as electro-surgical working elements;

FIG. 5 a perspective view of a surgical instrument having an end effector and an integrated manipulator to position the end effector;

FIG. 6 a perspective view of an end effector having three working elements;

FIG. 7 a sectional view of the end effector of FIG. 6;

FIG. 8 a perspective view of an end effector having four working elements;

FIG. 9 a perspective view of an end effector formed as a gripper in an opened state of the gripper;

FIG. 10 the end effector of FIG. 9 in a closed state of the gripper;

FIG. 11 a further embodiment of an end effector having an additional drive unit to rotate the end effector around its longitudinal axis; and

FIG. 12 a sectional view of the distal end of a surgical instrument having an end effector and a manipulator to position the end effector.

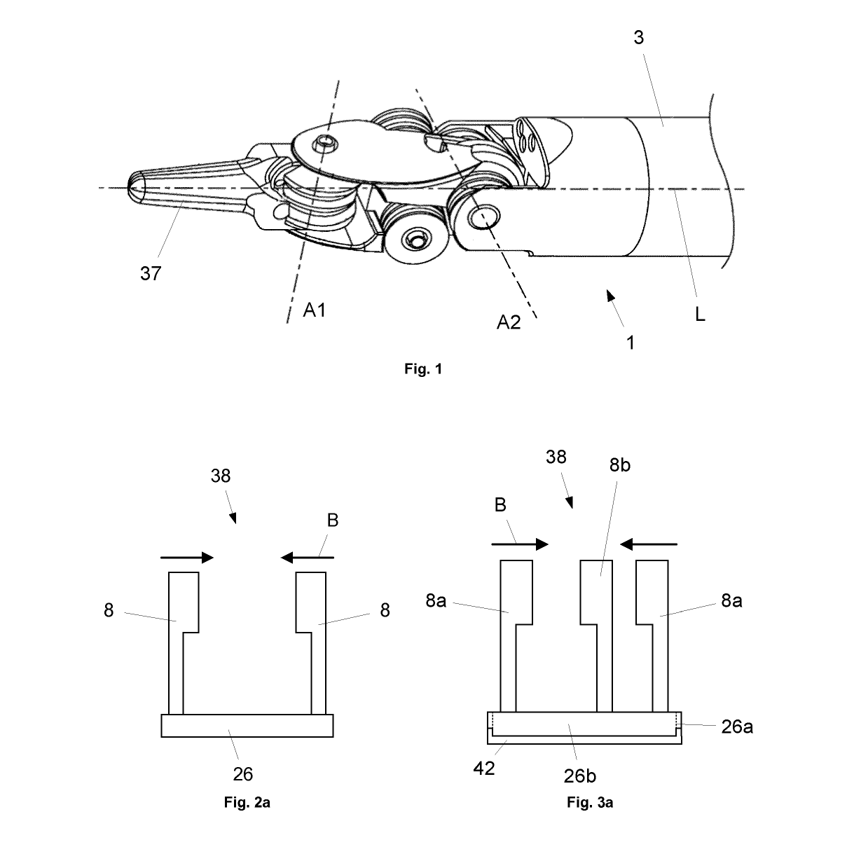

With regard to the explanation of FIG. 1, reference is made to the description introduction.

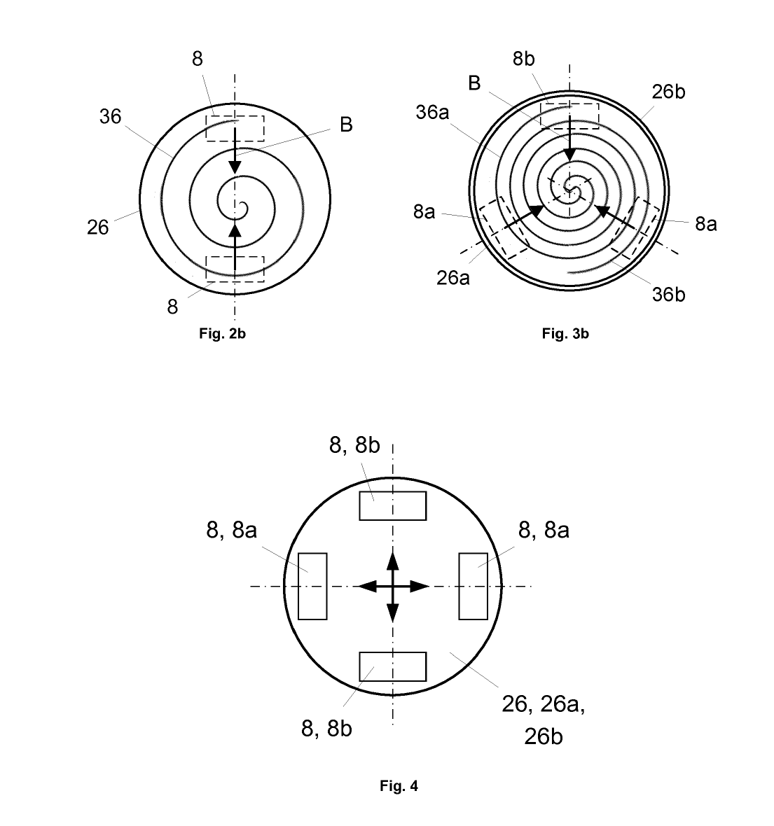

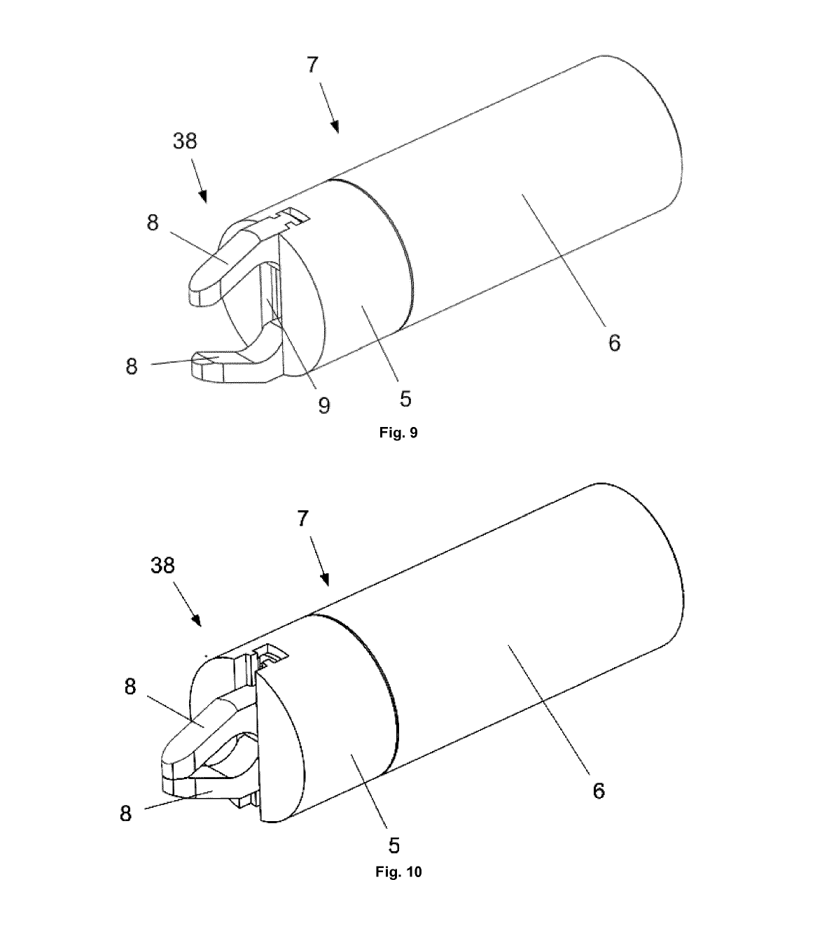

FIG. 2a shows a schematic side view of the tool 38 of an end effector 7, as is depicted by way of example in FIG. 9. The end effector 7 is, in this case, formed as a gripper having two working elements 8 arranged opposite each other. The two working elements 8 are guided on a proximal section within a guiding groove 9. At their proximal end (depicted at the bottom in the image), the working elements 8 engage with a rotatable element 26, which is part of a rotation-translation transmission 29. If the element 26 is driven in a rotational manner, the working elements 8 move towards each other or away from each other in the direction of the arrows B. A rotational movement of the element 26 is therefore translated into a purely translational movement of the working elements 8. The translational movement thereby occurs transversely to the rotational axis 10 of the element 26 in the radial direction.

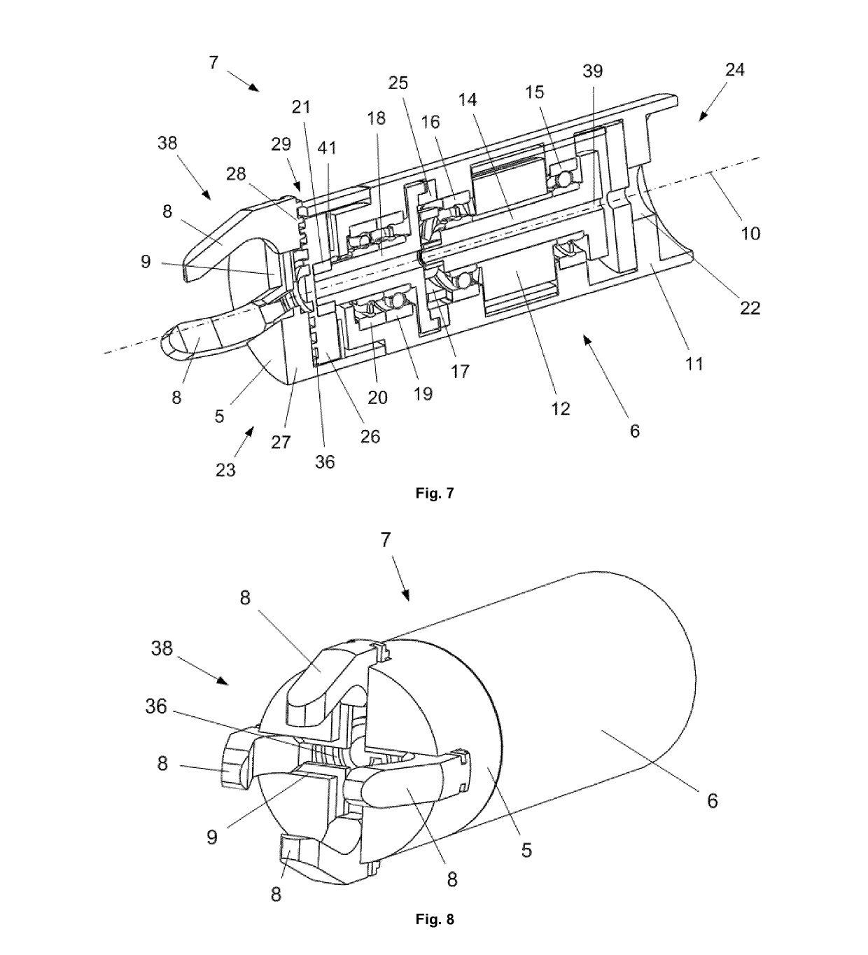

FIG. 2b shows a schematic view of the rotatable element 26. As can be recognised, the rotatable element 26 comprises a curve on its distal surface which engages with the working elements 8. The curve 36 is formed here as a spiral-shaped thread path, but, for example, could also be designed as a groove. The curve 36 spans a flat surface, the surface normal of which is directed in the direction of the rotational axis 10 (in FIG. 7), around which the element 26 rotates.

FIG. 3a shows a schematic side view of the tool 38 of an end effector 7 having three working elements 8, as is depicted by way of example in FIG. 6. The working elements 8 in turn engage with a rotatable element 26 of a rotation-translation transmission 29, wherein the working elements 8a engage with a first rotatable element 26a and the working element 8b with a second rotatable working element 26b. The rotatable elements 26a and 26b are able to rotate independently of each other and can, for example, be arranged nested one inside the other. By rotation of the elements 26a and 26b, the individual work elements 8a and 8b can therefore be moved towards each other or away from each other in the direction of the arrows B, independently of each other, depending on which rotatable element 26a and 26b rotates. The rotatable elements 26a and 26b can be driven by the same drive unit 6 or by different drive units. In a preferred embodiment of the invention, as shown in FIG. 3a, the rotation-translation transmission 29 comprises a clutch, in particular a double clutch, which is integrated functionally between the two rotatable elements 26a and 26b and can switch between the two elements 26a and 26b. Therefore, only one drive unit 6 is required in order to drive both rotatable elements 26a and 26b from the same drive unit 6. Depending on the switch state of the double clutch 42, both rotatable elements 26a and 26b can then be actuated either at the same time or alternately separately from each other. In other words, in the case of the latter variant, one rotatable element 26a or the other rotatable element 26b is therefore actuated.

FIG. 3b shows a schematised view of the rotatable elements 26a and 26b according to FIG. 3a. Each of the rotatable elements 26a and 26b has an associated curve 36a and 36b. One of the curves, for example 36a, serves to drive two of the three working elements, in particular the working elements 8a (e.g. the working element depicted on the left and right). The second curve, for example 36b, engages, however, with only one of the working elements, in particular working element 8b (e.g. the central working element). The individually driven working element 8b can therefore be driven independently of the two other working elements 8a. Such an embodiment of a gripper can, for example, be used to firstly roughly position an object and then to fix it by means of the third working element 8a. Another application could, for example, consist in gripping an object firstly by means of two working elements 8b and then implementing an electro-surgical operation by the third working element 8a being driven with temporal delay towards the object. The third working element 8a is, in this case, formed as an electro-surgical element, preferably typically as monopolar or bipolar HF tools to cut and coagulate body tissue.

FIG. 4 shows a further embodiment of a tool 38 of an end effector, as is depicted by way of example in FIG. 8. The end effector 7 comprises, in this case, four working elements 8 which are each arranged opposite each other in pairs. As explained above, the four working elements 8 are driven via a mutual rotatable element 26. Alternatively, the working elements 8 can be driven in pairs. In this case, the end effector 7 has two rotatable elements 26a and 26b, wherein two opposing working elements 8a engage with the first rotatable element 26a and the other two opposing working elements 8b with the second rotatable element 26b. Therefore, two of the working elements 8a, 8b are thereby each guided by a curve 36a or 36b. The movement direction of the two pairs runs exactly transversely to one another. In this embodiment, a first pair of working elements 8a, for example, is formed as a mechanical gripper. The pair of working elements 8b arranged transversely to this can, however, for example, be formed as an electro-surgical tool with which a current and/or a voltage can be transferred.

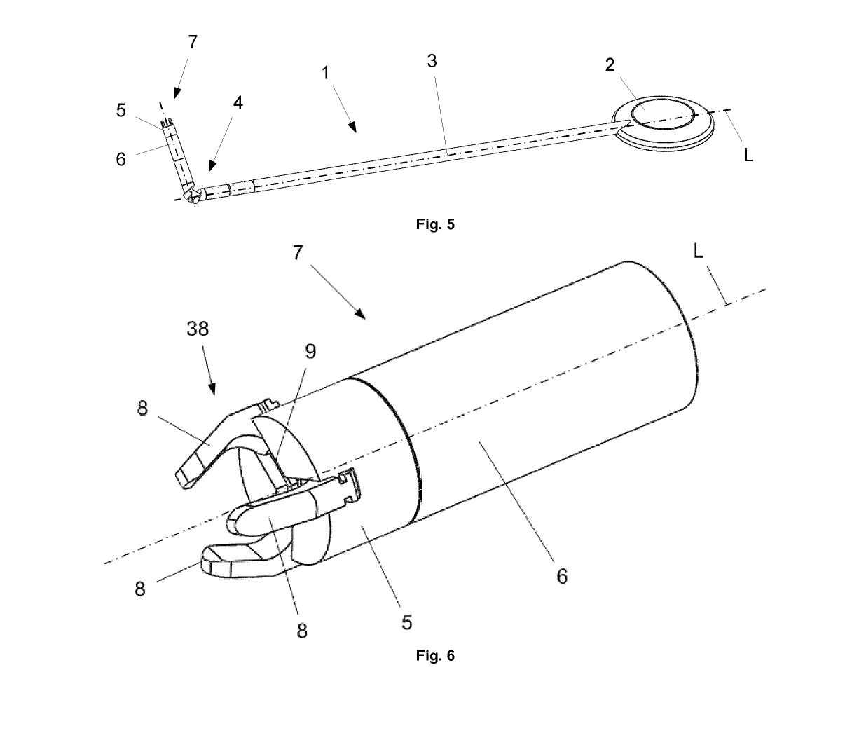

FIG. 5 shows a perspective view of a surgical instrument 1 for minimally invasive surgery, which is provided for fastening to a surgery robot. The surgical instrument 1 comprises a fastening device 2 on its proximal end (shown on the right-hand side of the image), with which it can be fastened to a surgery robot or to an instrument holder.

The surgical instrument 1 depicted in FIG. 2 furthermore comprises a shaft 3 running in a longitudinal direction L, at the distal end of which (depicted on the left in the image), a manipulator 4 for positioning an end effector 7 is arranged.

The surgical instrument 1 can, for example, be a gripping, holding, cutting, sawing, grinding, connecting or joining instrument or any other surgical instrument. The end effector 7 of the surgical instrument 1 can, for example, be formed as a scalpel, scissors, tongs, a trocar, etc. The use of optical or image-processing tools, such as, for example, lamps, laparoscopes or cameras, is also possible.

FIG. 6 shows a perspective view of an end effector 7 according to a first embodiment of the invention. The end effector 7 comprises a drive unit 6, on the distal end of which (depicted on the left in the image) the actual tool 38 is provided. The end effector 7 is formed here as a gripper having three working elements 8 or grippers. Each working element 8 is guided in a groove 9 and can execute a translational movement in the radial direction during actuation of the end effector 7. The three working elements 8 are arranged here at an angle to one another, preferably each at a 120° angle.

The tool 38 of the end effector 7 is fastened releasably to the drive unit 6. To fasten the tool 38, a fastening device 5 is provided on the proximal end of the tool 38. The fastening device 5 can, for example, comprise a screw, plug or catch connection. With the aid of the fastening device 5, it is possible to exchange the tool 38 quickly and simply for another or to replace it if needed. It is therefore no longer required to exchange the entire surgical instrument 1.

Alternatively, the tool 38 could naturally also be formed as a unit together with the drive unit 6. In this case, a corresponding fastening device could be provided on the proximal end of the drive unit 6.

FIG. 7 shows a sectional view of the end effector 7 of FIG. 6. The end effector 7 comprises substantially two units which are connected to each other releasably, in particular a drive unit 6 and the tool 38 fastened to the distal end of the drive unit 6. The drive unit 6 comprises, in this exemplary embodiment, an electric motor 12 which drives a shaft 14 in a rotational manner. The shaft 14 is thereby mounted rotatably in a housing 11 of the drive unit 6 with the aid of two ball bearings 15, 16. The drive unit 6 furthermore comprises a transmission 17 which transfers the rotational movement executed by the shaft 14 to an output shaft 18. The output shaft 18 is likewise mounted in the housing 11 of the drive unit 6 using two ball bearings 19, 20.

A sprocket 21 is located on the free end of the output shaft 18, said sprocket being plugged into a corresponding recess of a rotation-translation transmission 29. Alternatively, any other known device for torque transfer could, of course, be provided in which the torque exerted by the shaft 18 driven by the electric motor is transferred directly to the tool 38, such as, for example, a shaft hub connection.

The rotation-translation transmission depicted in FIG. 7 comprises a rotatable element 26 connected non-rotatably to the shaft 18, said rotatable element here being formed to be disc-shaped and having a planar curve 36 on its surface pointing in the distal direction, said planar curve engaging with the working elements 8 or grip jaws of the tool 38. In the case of a rotational movement of the output shaft 18 driven by the electric motor 12, the rotatable element 26 likewise rotates around the rotational axis 10. This rotational movement is then transferred via the rotation-translation transmission 29 to the gripper 8, such that these are moved towards each other or away from each other in a purely translational manner. The grip jaws 8 are thereby guided within grooves 9 which run substantially in a radial direction.

A plug connection is provided here to fasten the tool 38 to the distal end of the drive unit 6. The tool 38 comprises, for this purpose, a fastening section 5 which can be plugged onto the distal end of the drive unit 6. The fastening section 5 preferably comprises catch means (not shown) to latch with the drive unit 6.

The drive unit 6 depicted in FIG. 7 furthermore comprises a brake 25 to brake a drive movement. Furthermore, a continuous channel 39 runs within the drive unit 6, through which a medium, for example air or a liquid, such as, for example, a salt solution, can be conducted. The channel 39 runs through the shafts 14, 18. The shafts 14, 18 therefore have a hollow interior. The tool 38 has a passage opening corresponding to the channel 39, through which the medium can be conducted to the operation location. During an operation, the medium is preferably introduced into the channel 39 with a pressure p1 which is greater than the pressure p2 prevailing in the patient.

The end effector depicted in FIG. 7 additionally has a repelling component 41 which acts on the rotating element 26 and is supported on the housing inner wall of the fastening section 5. The repelling component 41 is formed here as a spiral spring and is tensioned if the rotating element 26 is actuated in the rotational direction to open the working elements 8. The spiral spring 41 can also be pre-tensioned such that it applies a repelling force to the rotating element 26 even in the closed state of the gripper. Due to the repelling force, the working elements 8 are driven in the closing direction (arrow direction B, see FIGS. 2a and 3a). Since the spring force of the spring 41 acts in the closing direction, during opening of the end effector it acts against the torque of the drive unit 6. During closing of the end effector, the spring force, however, interacts with the torque of the drive unit 6. The spring force is preferably selected in such a way that the end effector 7 can close automatically in the event of failure of the drive unit 6. However, the spring is preferably designed in such a way that it only closes the working elements 8 to the extent that the working elements 8 no longer project radially over the fastening section 5, as shown in FIG. 8 or FIG. 11, but rather close at least flush, as for example shown in FIG. 9. In other words, the working elements 8 are not completely closed by the repelling force of the spring. Therefore, it can be prevented that the end effector 7 grips an object in an undesired manner in the event of failure of the drive unit 6 due to the automatic closing movement.

At this point, it should be noted that the invention is not limited to the use of a spring, but alternatively other components can be used as a repelling component 41, which cause an automatic closing of the working elements 8.

FIG. 8 shows a perspective view of an end effector 7 having a tool 38 which comprises four working elements 8. The working elements 8 are, in this case, driven in pairs by different curves 36a, 36b. Therefore, for example, the two horizontally depicted working elements 8 can engage with a first curve 36a, and the two vertically depicted working elements 8 with a second curve 36b. Depending on the design of the curves 36a, 36b, it is therefore possible to drive the two gripper pairs at different speeds. In this exemplary embodiment, a clutch 42 is preferably built in, as is shown in FIG. 3a. Therefore, the curves 36a, 36b can be driven together or separately from each other depending on the switch state of the clutch. The working elements 8 can be provided analogously to FIG. 4 with different functions. Therefore, a pair of working elements can be formed specifically for electro-surgical interventions.

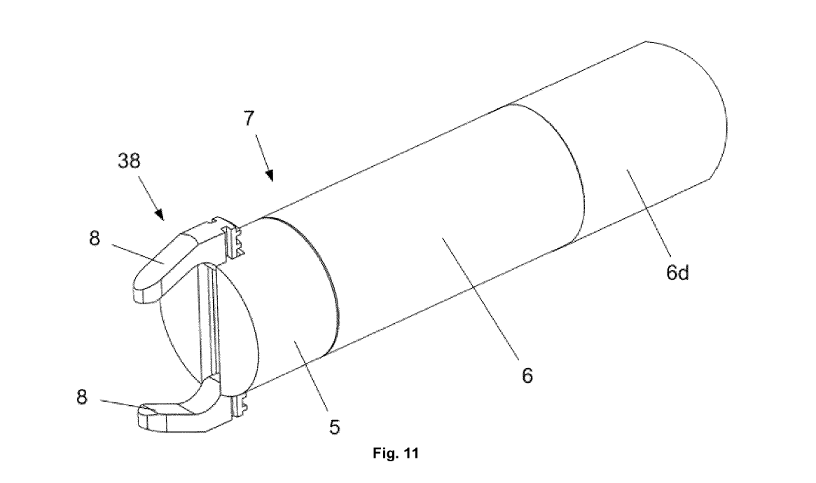

FIG. 9 shows a further embodiment of an end effector 7 formed as a gripper, the tool 38 of which has two gripper elements 8 arranged opposite each other which are guided in a groove 9 at their proximal end. The gripper elements 8 can in turn be moved towards each other or away from each other by a rotational drive movement of the electric motor 12. In FIG. 9, the opened state is depicted, and in FIG. 10 the closed state.

FIG. 11 shows a further embodiment of an end effector 7 having two grip elements 8. Contrary to the embodiment of FIGS. 9 and 10, this end effector 7, however, comprises an additional drive unit 6d, which is arranged at the proximal end of the drive unit 6. The additional drive unit 6d serves in this case to rotate the tool 38 around the longitudinal axis 10 of the end effector 7. The additional drive unit 6d is preferably formed to be identical to the drive unit 6 and engages with a recess 22 provided at the proximal end 24 of the drive unit 6 with its distal end (see FIG. 7). Therefore the drive units can be coupled to each other with a shaft hub connection, wherein the shaft 18 or the sprocket 21 of one drive unit 6d can be connected to the hub 22 of the other drive unit 6 which is integrated into the housing 11. In the case of an actuation of the electric motor 12 of the drive unit 6d, the torque is then transferred to the drive unit 6 and the tool 38 which therefore rotate together around the axis 10. The two drive units 6 and 6d are preferably connected to each other releasably, but can also be connected to each other in a fixed manner.

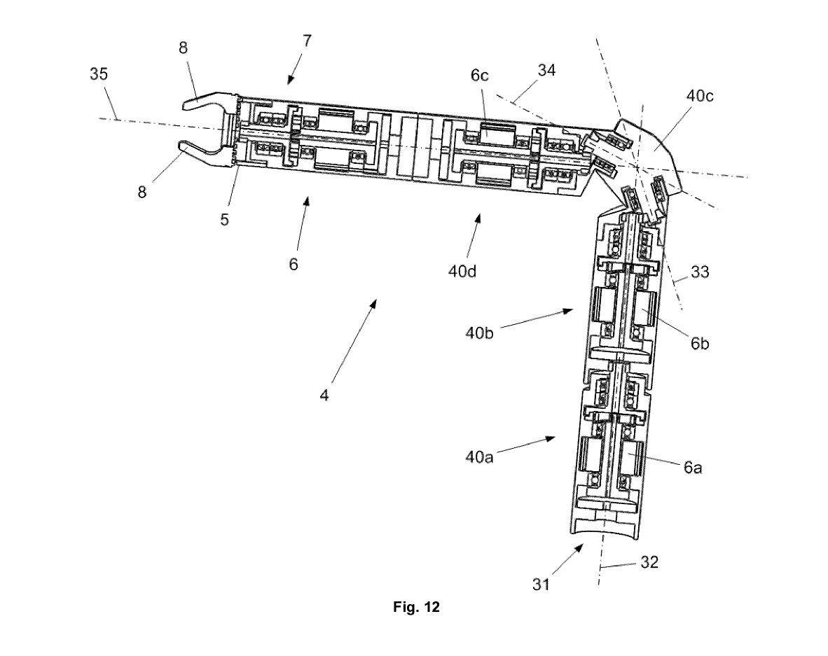

FIG. 12 shows a sectional view of the distal end of a surgical instrument 1 having an end effector 7 and a manipulator 4 to position the end effector 7. The manipulator 4 thereby comprises the elements 40a-40d. The proximally arranged element 40a can be fastened to the shaft 3 of a surgical instrument 1. For the purpose of the fastening, a screw, plug or catch connection, for example, or any other known connection mechanism can be used. The proximal end 31 is connected non-rotatably to the shaft 3 during the operation.

The element 40a comprises, in this embodiment, a drive unit 6a, as is depicted by way of example in FIG. 7. The drive unit 6a thereby serves to drive a first rotatable element 40b, which is arranged on the distal end of the element 40a. The two elements 40a, 40b are preferably connected to each other via a plug connection.

The first rotatable element 40b is rotatable around a first rotational axis 32 which runs in the longitudinal direction L of the shaft 3. A distally connected second rotatable element 40c is rotatable around a second rotational axis 33 relative to the element 40b, said second rotational axis being inclined compared to the first rotational axis 32 at a predetermined angle. A third rotatable element 40d which is distally adjacent to the element 40c is rotatable around a third rotational axis 34 compared to the element 40c, said third rotational axis being inclined compared to the rotational axis 35 at a second angle. The two angles are preferably of the same size, but can also be of different sizes.

The individually rotatable elements 40b-40d are each driven in a rotational manner by an associated drive unit 6a-6c. The first drive unit 6a to drive the first rotating element 40b is thereby integrated into element 40a.

The drive unit 6b to drive the second rotatable element 40c in a rotational manner is arranged in the first rotatable element 40b. The third drive unit 6c to drive the third rotatable element 40d in a rotational manner is accommodated in the third rotatable element 40d. A drive unit is not provided in the second rotatable element 40c in this variant.

By actuating the first drive unit 6a, the first rotatable element 40b rotates around the first rotational axis 32. If the second drive unit 6b is driven, the second rotatable element 40c rotates around the second rotational axis 33. By actuating the third drive unit 6c, the third rotatable element 40d finally rotates around the third rotational axis 34.

By driving the second and third rotatable element 40c and 40d in a rotational manner around their rotational axes 33, 34, the end effector 7 connected to the distal end of the manipulator 4 can be unwound at a determined angle relative to the longitudinal axis L of the surgical instrument. This angle thereby corresponds to double the sum of the two angles, around which the rotational axes 33 and 34 are inclined relative to the longitudinal axis L or rotational axis 35. If the two angles, for example, each amount to 22.5 degrees, the end effector 7 can be deflected by up to 90 degrees. Depending on the design of the rotational axes 33, 34, larger or smaller angles can of course also be achieved.

As can furthermore be recognised in FIG. 12, the drive units 6a-6c are all constructed identically. As explained above, the optionally present drive unit 6d can also be constructed identically to the drive units 6a-6c. Likewise, the drive unit 6 can be constructed identically to the drive units 6a-6d. The surgical instrument 1 can therefore be produced particularly simply and cost effectively.

1. End effector for a surgical instrument, comprising:
a drive unit having an electric motor which drives a shaft in a rotational manner,
a rotation-translation transmission connected to the shaft which translates a rotational movement of the shaft into a translational movement; and
several working elements of the end effector which are coupled to the rotation-translation transmission and are driven by this in a translational manner,
wherein the end effector comprises a repelling component which drives the working elements in a closing direction.

2. End effector according to claim 1, wherein the translational movement runs transversely to a rotational axis around which the shaft rotates.

3. End effector according to claim 1, wherein the rotation-translation transmission comprisesa rotating element having a planar curve which engages with the working element driven in a translational manner and guides this.

4. End effector according to claim 3, wherein the curve is designed as a spiral-shaped thread path or as a spiral-shaped groove.

5. End effector according to claim 3, wherein the curve spans a planar surface, the surface normal of which is directed in the direction of the rotational axis of the shaft.

6. End effector according to claim 1, wherein the repelling component is formed as a spring and acts on the rotation-translation transmission.

7. End effector according to claim 3, wherein the rotation-translation transmission comprises several curves which each engage with at least one working element.

8. End effector according to claim 7, wherein the rotation-translation transmission comprises several rotatable elements having a corresponding curve which are actuated independently of one another.

9. End effector according to claim 8, wherein the rotation-translation transmission comprises a clutch which switches between the two rotatable elements.

10. End effector according to claim 3, wherein the shaft of the drive unit engages with at least one rotating element of the rotation-translation transmission.

11. End effector according to claim 1, wherein the end effector comprises at least two working elements which are arranged opposite each other or at an angle to each other and can be moved towards each other or away from each other by a rotational movement of the shaft.

12. End effector according to claim 11, wherein at least one working element is formed as an electro-surgical working element.

13. End effector according to claim 1, wherein the end effector has a fastening device with which the tool of the end effector can be fastened releasably to the drive unit.

14. End effector according to claim 1, wherein the end effector has a fastening device with which the end effector, together with its drive unit, can be fastened to the shaft of a surgical instrument.

15. End effector according to claim 1, wherein the end effector comprises a second drive unit with which the end effector can be rotated around the rotational axis of the shaft.

16. End effector according to claim 15, wherein the drive unit to actuate the drive element of the end effector is constructed identically to the second drive unit to rotate the end effector.

17. End effector according to claim 1, wherein the drive unit comprises an electric motor.

18. End effector according to claim 1, wherein the drive unit comprises a transmission.

19. Surgical instrument for use in minimally-invasive surgery, having a shaft which extends in a longitudinal direction, wherein an end effector according to claim 1 is provided on the shaft.
